(19) United States
(12) Patent Application Publication
(10) Pub. No.: US 2022/0343200 A1
(43) Pub. Date: Oct. 27, 2022

(54) PARAMETER ESTIMATION DEVICE, PARAMETER ESTIMATION METHOD, AND PARAMETER ESTIMATION PROGRAM

(71) Applicant: NIPPON TELEGRAPH AND TELEPHONE CORPORATION, Tokyo (JP)

(72) Inventors: Masahiro KOJIMA, Tokyo (JP); Takeshi KURASHIMA, Tokyo (JP); Hiroyuki TODA, Tokyo (JP)

(73) Assignee: NIPPON TELEGRAPH AND TELEPHONE CORPORATION, Tokyo (JP)

(21) Appl. No.: 17/763,634

(22) PCT Filed: Oct. 2, 2019
(86) PCT No.: PCT/JP2019/038929

§ 371 Date: Mar. 24, 2022

(51) Int. Cl. G06N 7/00

(52) U.S. Cl. CPC G06N 7/005; G16H 50/20

(57) ABSTRACT

Markov chain parameters can be accurately estimated using transition data whose observation interval is not constant. Assuming that transition intervals of a Markov chain defined from a set of states are steps, input data that is transition data including the number of transitions between states in a set of transitions between states is received, and a parameter relating to a model regarding the number of steps representing a probability that a transition of a predetermined number of steps occurs from each state and a parameter relating to a model regarding a transition probability representing a probability that a one-step transition occurs from each state, the models being models for the number of transitions between the states of the input data, are estimated such that an objective function including a term for a generation probability of the input data given by the model regarding the number of steps and the model regarding the transition probability is optimized.

### TECHNICAL FIELD

The disclosed technology relates to a parameter estimation apparatus, a parameter estimation method, and a parameter estimation program.

### BACKGROUND ART

A Markov process is a versatile model capable of expressing various dynamic systems and is used for various purposes such as analysis of urban people and traffic flow and analysis of queuing at ticket sales counters.

For example, a method of estimating Markov chain parameters from complete one-step transition data between states in a set of states has been shown as a conventional technique (see NPL 1).

### CITATION LIST

### Non Patent Literature

NPL 1: Patrick Billingsley. Statistical methods in Markov chains. The Annals of Mathematical Statistics, pp. 12-40, 1961.

### SUMMARY OF THE INVENTION

### Technical Problem

However, data collected in a real environment is not step-by-step data, but multi-step data whose observation interval is not a fixed number of steps. In existing methods, parameters of an original one-step Markov chain cannot be estimated from such multi-step transition data. This is because a transition probability that multi-step transitions follow and a transition probability that one-step transitions follow are different and thus it is necessary to consider the difference between the two.

The disclosed technology has been made in view of the above points and it is an object of the disclosed technology to provide a parameter estimation apparatus, a parameter estimation method, and a parameter estimation program that can accurately estimate Markov chain parameters using transition data whose observation interval is not constant.

### Means for Solving the Problem

A first aspect of the present disclosure is a parameter estimation apparatus including an estimation unit configured to, assuming that transition intervals of a Markov chain defined from a set of states are steps, receive input data that is transition data including the number of transitions between states in a set of transitions between states and estimate a parameter relating to a model regarding the number of steps representing a probability that a transition of a predetermined number of steps occurs from each state and a parameter relating to a model regarding a transition probability representing a probability that a one-step transition occurs from each state, the models being models for the number of transitions between the states of the input data, such that an objective function including a term for a generation probability of the input data given by the model regarding the number of steps and the model regarding the transition probability is optimized.

A second aspect of the present disclosure is a parameter estimation method including a computer executing a process including, assuming that transition intervals of a Markov chain defined from a set of states are steps, receiving input data that is transition data including the number of transitions between states in a set of transitions between states and estimating a parameter relating to a model regarding the number of steps representing a probability that a transition of a predetermined number of steps occurs from each state and a parameter relating to a model regarding a transition probability representing a probability that a one-step transition occurs from each state, the models being models for the number of transitions between the states of the input data, such that an objective function including a term for a generation probability of the input data given by the model regarding the number of steps and the model regarding the transition probability is optimized.

A third aspect of the present disclosure is a parameter estimation program causing a computer to, assuming that transition intervals of a Markov chain defined from a set of states are steps, receive input data that is transition data including the number of transitions between states in a set of transitions between states and estimate a parameter relating to a model regarding the number of steps representing a probability that a transition of a predetermined number of steps occurs from each state and a parameter relating to a model regarding a transition probability representing a probability that a one-step transition occurs from each state, the models being models for the number of transitions between the states of the input data, such that an objective function including a term for a generation probability of the input data given by the model regarding the number of steps and the model regarding the transition probability is optimized.

### Effects of the Invention

According to the disclosed technology, Markov chain parameters can be accurately estimated using transition data whose observation interval is not constant.

### BRIEF DESCRIPTION OF DRAWINGS

FIG. 1 is a diagram illustrating an example of ideally recorded data for a movement history.

FIG. 2 is a diagram illustrating an example of actually collected data for a movement history, whose observation interval is not constant.

FIG. 3 is a diagram illustrating a Markov chain expressing a monthly transition probability of symptoms of a disease.

FIG. 4 is a diagram illustrating an example of data recorded for a medical treatment history at an ideal frequency.

FIG. 5 is a diagram illustrating an example of data recorded for a medical examination history at a frequency of actual observation, whose observation interval is not constant.

FIG. 6 is a diagram when data having information regarding the number of steps is taken as an input.

FIG. 7 is a diagram when data having no information regarding the number of steps is taken as an input.

FIG. 8 is a block diagram illustrating a configuration of a parameter estimation apparatus of the present embodiment.

FIG. 9 is a block diagram illustrating a hardware configuration of the parameter estimation apparatus.

FIG. 10 is a flowchart showing a flow of a parameter estimation process performed by the parameter estimation apparatus.

### DESCRIPTION OF EMBODIMENTS

Hereinafter, examples of embodiments of the disclosed technology will be described with reference to the drawings. The same or equivalent components and parts are denoted by the same reference signs in each drawing. The dimensional ratios in the drawings are exaggerated for convenience of explanation and may differ from the actual ratios.

In the following, first, the background and outline of the present disclosure will be described and then principles and an optimization method according to the present disclosure will be described.

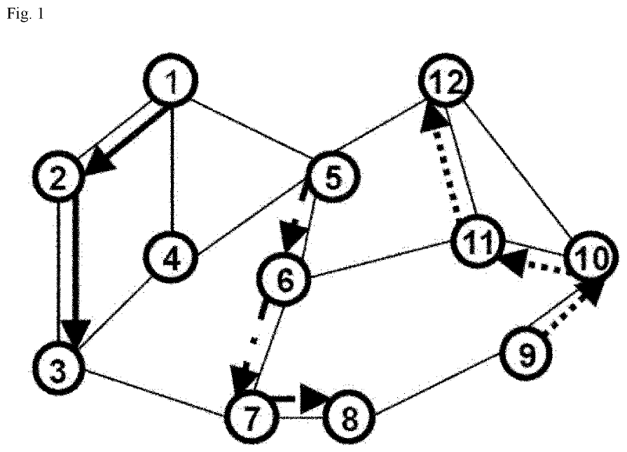

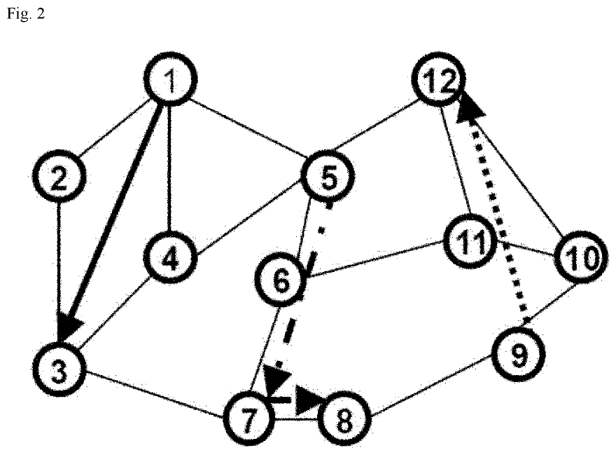

Regarding the background, matters related to the nature of a Markov process will be described. Because a transition probability that is a parameter of the Markov process is generally unknown, it is necessary to estimate the parameter from observation data. FIG. 1 is a diagram illustrating an example of ideally recorded data for a movement history. FIG. 2 is a diagram illustrating an example of actually collected data for a movement history, whose observation interval is not constant. States as illustrated in FIG. 1 correspond, for example, to areas of a city. If an ideal movement history of observing each transition between states can be used as input data, a transition probability can be easily estimated based on the number of transitions between states (see NPL 1). However, data collected in a real environment has the property that the observation interval is not constant due to the difficulty of comprehensive data acquisition caused by data processing for personal information protection, restrictions on data collection opportunities, and the like. While transition data in an ideal condition is assumed to be one-step transition data that is recorded step by step, input data that can be actually acquired is expressed as multi-step transition data that is transition data in which only the first and last states of a “transition consisting of a plurality of transitions that have occurred between states” are recorded. That is, when applied to such a case, a Markov chain is expressed as a multi-step Markov chain whose observation interval is not constant rather than as an ideal step-by-step Markov chain. In the following, transition intervals of a Markov chain defined from a set of states are treated as steps. As is known with respect to Markov chains, transitions between states here include not only those to other states but also those remaining in the same states. In the following, a step-by-step Markov chain is also simply referred to as a one-step Markov chain.

The following two are examples of data in which only the first and last states of a transition consisting of a plurality of transitions are recorded. The first is transition data of movement histories provided by a mobile phone company or the like and obtained by converting GPS data of people in areas. In such transition data, only histories of transitions between areas where users have stayed for a certain period of time or longer are recorded in order to protect personal information and reduce the volume of data. Thus, even if a transition consisting of two transitions of states 1 →2 →3 actually occurs as shown by solid arrows in FIG. 1, actually recorded transition data is not so. For example, when areas where users have stayed for a certain period of time or longer are states 1 and 3, only the two states before and after a transition consisting of two transitions are given as transition data like states 1 →3 as illustrated in FIG. 2. Only data before and after a transition consisting of three transitions may sometimes be given as shown by dotted arrows in FIG. 2. Thus, this data is expressed as multi-step transition data in which only the two states before and after a transition consisting of a plurality of transitions are recorded.

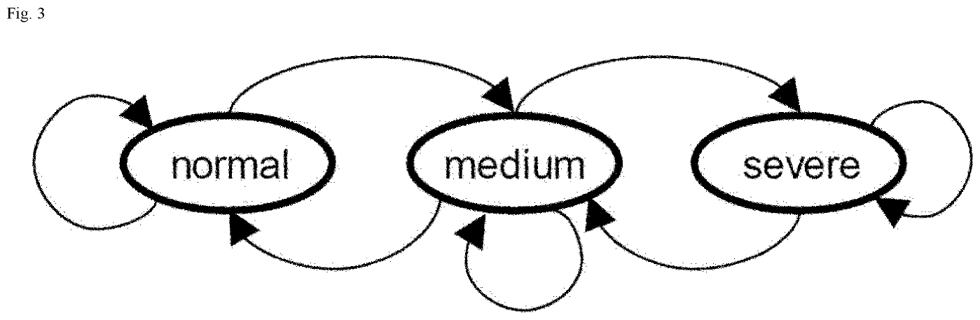

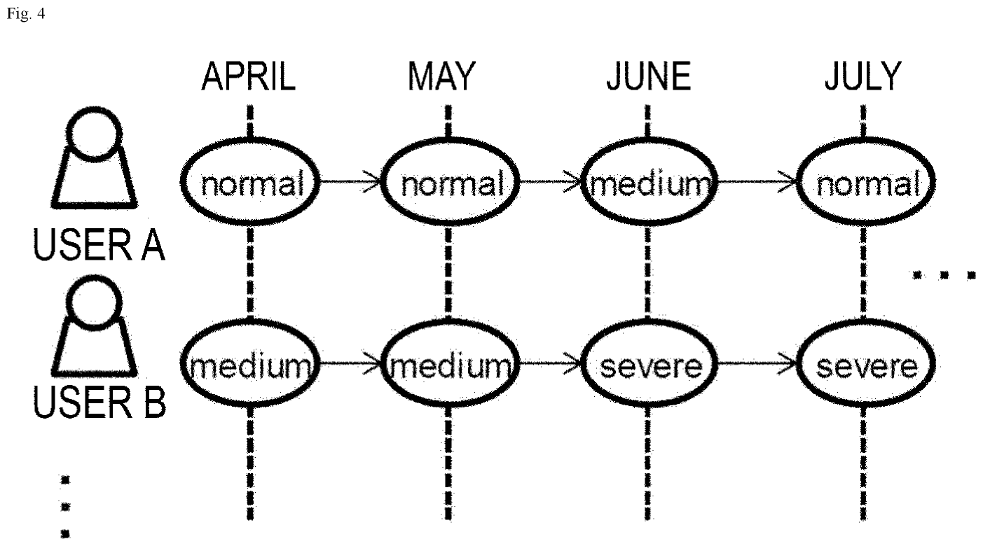

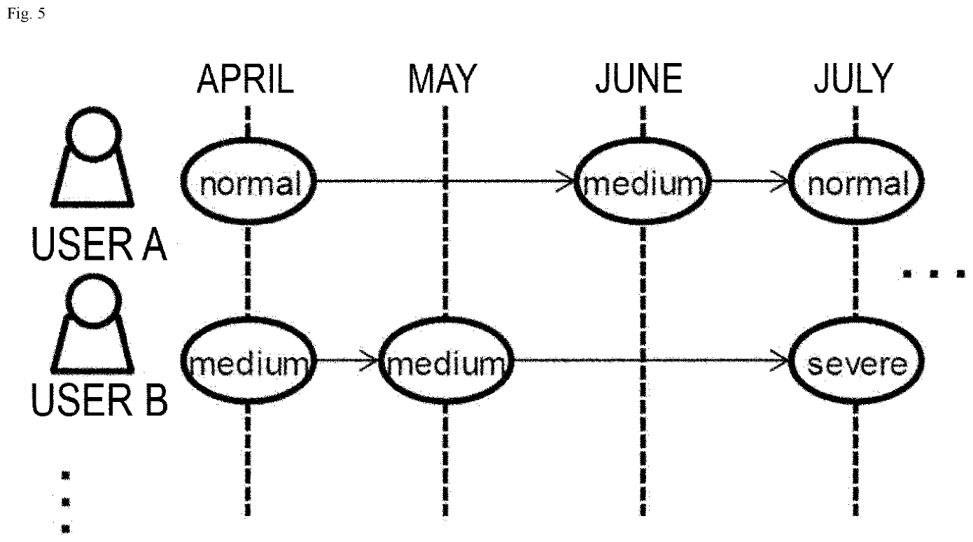

The second example is monthly transition data of medical treatment histories held by medical institutions such as hospitals. FIG. 3 is a diagram illustrating a Markov chain expressing a monthly transition probability of symptoms of a disease. FIG. 4 is a diagram illustrating an example of data that is recorded for a medical treatment history at an ideal frequency. FIG. 5 is a diagram illustrating an example of data recorded for a medical examination history at a frequency of actual observation, whose observation interval is not constant. A case where a monthly transition probability of symptoms of a disease is expressed by a Markov chain as illustrated in FIG. 3 will be considered. Normal, medium, and severe indicate that the symptoms are normal, slightly worse, or worse, respectively. However, medical treatment history data held by medical institutions is considered data whose observation interval is not constant such that there are months when patients cannot visit due to their circumstances as illustrated in FIG. 5, rather than data on patients who always visit regularly every month as illustrated in FIG. 4. Thus, this data is also expressed as multi-step transition data in which only the two states before and after a transition consisting of a plurality of transitions are recorded, similar to the first data.

Therefore, a method of estimating parameters of an original one-step Markov chain from multi-step transition data is proposed in the method of the present disclosure. The point of the present disclosure is to use a method of constructing a transition probability of a plurality of steps, that is, a multi-step transition probability, from a transition probability of a one-step Markov chain. The configuration and operation of the present disclosure will be described below after a model of a Markov chain and an objective function of a multi-step transition probability are described.

Preliminary

A set of states is represented as shown below. This will also be simply referred to as a state set X in the following description.

X={1,2, . . . ,|X|}

A Markov chain in discrete time on the state set X is defined as a stochastic process {Xₜ; t=1, 2, . . . } having the Markov property shown in the following expression (1).

[Math. 1]

Pr(Xₜ₊₁=xₜ₊₁|Xₖ=xt;k=0, . . . ,t)=Pr(Xₜ₊₁=xₜ₊₁|Xₜ=xₜ)(∀xₖ∈X,∀t∈ [symbol] _{≥0}) (1)

The Markov chain can be defined as a triad of {X, P, q}. A function P: X×X →[0,1] defined by the following expression (2) is called a one-step transition probability.

[Math. 2]

[symbol]

(xₙₑₓₜ|x) [symbol] Pr(Xₜ₊₁−xₙₑₓₜ|Xₜ=x) (2)

A matrix representation of this transition probability is expressed as P, (P)xx′=P(x′|x).

A theorem for multi-step transition probabilities is shown below.

Theorem 1

The probability of an m-step transition is given by a transition probability matrix P to the mth power (see Reference 1 (e.g., Theorem (2.1)).

[Reference 1] Richard Durrett, Norio Konno (translator), Kazutaka Nakamura (translator), Takahiro Some (translator), and Ma Kasumi (translator). Essentials of Stochastic Processes. Springer Fairlark Tokyo, 2005.

From this theorem, it can be seen that the probability of a two-step transition is P² and the probability of a three-step transition is P³. The proof that this theorem holds can be confirmed from the viewpoint that expression (4) holds if n=1 in the Chapman-Kolmogorov equation of the following expression (3).

[Math. 3]

(P^{m+n})ᵢⱼ=Σₖ(P^{m})ᵢₖ(Pⁿ)ₖⱼ (3)

(P^{m+1})ᵢⱼ=Σₖ(P^{m})ᵢₖ(P)ₖⱼ (4)

Next, a model and an optimization method used for an objective function of the present disclosure will be described based on the above principles.

Model

The method of the present disclosure is a method of estimating parameters of an original one-step Markov chain from multi-step transition data. An approach of constructing a probabilistic model and estimating its parameters from data will be adopted for this purpose. The model constructed in the method of the present disclosure includes two models, (i) a model regarding the number of steps f(k|λᵢ) representing the probability of a k-step transition from each state i and (ii) a model regarding a transition probability P_{η} representing the probability of a one-step transition from each state. λ={λᵢ}_{i∈X} are parameters to be estimated. These parameters are collectively expressed as θ={λ|η}. FIG. 4 is a diagram of a probability distribution showing how many steps a transition is likely to occur through from each state. FIG. 5 is a diagram representing one-step transition probabilities. FIG. 4 illustrates an image of the model regarding the number of steps and FIG. 5 illustrates an image of the model regarding the transition probability. For example, a Poisson distribution of the following expression (5) can be used for the model f(k|λᵢ) regarding the number of steps k.

[Math. 4]

[symbol]

(k|λᵢ)=exp{−∥ᵢ+k log(λᵢ)−log Γ(k+1)} . . . (5)

Of course, a discrete probability distribution such as a categorical distribution, a geometric distribution, a zero truncated Poisson distribution (Zero Truncated Poisson: ZTP), and a negative binomial distribution as well as that of expression (5) can be used for the model f(k|λᵢ) regarding the number of steps k. If properly normalized, a continuous probability distribution such as an exponential distribution can also be used for the model f(k|λᵢ) regarding the number of steps k. A distribution belonging to an exponential family of distributions can also be used for the model f(k|λᵢ) regarding the number of steps k. Any probability distribution other than the examples given here can be used for the model f(k|λᵢ) regarding the number of steps k. Even when the number of possible steps is limited (for example, when the probability that k >Kmax is 0, letting Kmax represent a maximum value of the number of steps), this can be dealt with by using a truncated distribution of an original distribution to be used. Regarding the method of constructing a truncated distribution, for example, see Reference 2 which shows an example of constructing a truncated normal distribution from a normal distribution. [Reference 2] NL Johnson, Samuel Kotz, and NBalakrishnan. Continuous Univariate Probability Distributions, (Vol. 1). John Wiley & Sons Inc., NY, 1994.

Even if the number of steps that can occur explicitly is not limited, it is possible to construct a model that approximates an infinite sum without limiting the number of steps by using the following property under the following condition. The “following condition” is a condition that a Markov chain having the transition probability P_{η} is irreducible and aperiodic for any parameter The “following property” is that the transition probability to the mth power converges to a steady distribution at the limit of m (see Reference 1 (e.g., Theorem (4.5)). Specifically, a term which expresses the transition probability by a steady distribution representing all k's for which k >K^{tr} is constructed after a sufficiently large threshold value Kₜᵣ is set. When this method is used, for example, Equation (13) which will be described later can be expressed approximating an infinite sum over the number of steps k by a finite sum as shown on the right side of the following expression.

Pr(Nᵢⱼ|θ)≈(Σₖ₌₀^{K}^{ₜᵣ}f(k|λᵢ)(Pᵢⱼ^{k})ᵢⱼ+{1−F(Kₜᵣ|λᵢ)}(π_{η})ⱼ)^{N}^{ᵢⱼ}

where F(k|λᵢ) is the cumulative probability distribution of the model F(k|λᵢ), π_{η} is the steady-state probability of the Markov chain having the transition probability P_{η}, and symbol
≈
indicates that the right side of the expression approximates the left side.

The following expression (6), which is a model in which different parameters are given for transition probabilities between states, may be used for the model P_{η} regarding the transition probability.

[Math. 5]

P_{η}={ηᵢⱼ}_{i∈xj∈Ω} (6)

A model such as that of the following expression (7) based on a logistic regression model with a parameter η={v^{base}, v^{ftr}} may also be used.

$$\begin{matrix}[ Math . 6 ] & \\ {( {P}_{η} )}_{ij} = \{ \begin{matrix}exp \{ g ( i , j ; η ) \} / {\sum }_{k ∈ {Ω}_{i}} exp \{ g ( i , k ; η ) \} & ( j ∈ {Ω}_{i} ) \\ 0 & ( otherwise )\end{matrix} & ( 7 )\end{matrix}$$

Where Ωᵢ is a set of states that can be reached from state i in one step, g(i, j, η) is a score function defined such that g(i, j, η)=vᵢⱼ^{base}+φ(i,j)Tᵥ^{ftr}, and φ(i,j) is a feature vector. v^{base} is a parameter regarding the state transition and v^{ftr}r is a parameter regarding the feature vector. The feature vector ϕ(i,j) is a vector having arbitrary attribute information regarding states i and j. For example, in the case of a movement history, the feature vector ϕ(i,j) is a vector with each element representing a geographical distance between states or the like. In the case of a medical examination history, the feature vector ϕ(i,j) is a vector with each element representing the degree of similarity between the user's health conditions. Any other models that can express the transition probability may be used. If the parameter η can be estimated, the transition probability of the original one-step Markov chain can also be estimated by the model Pr_{η}.

Problem Setting

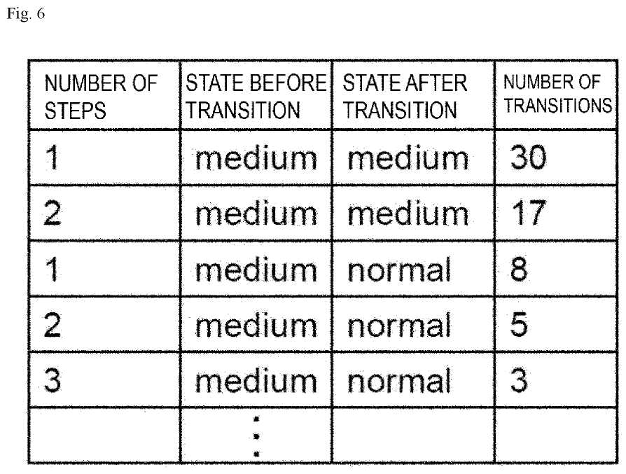

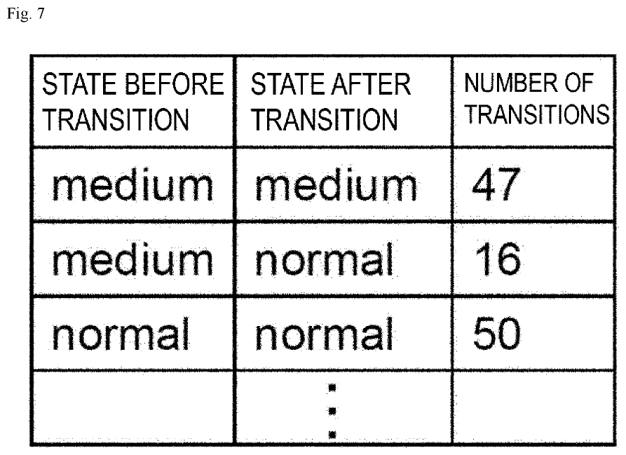

The following two settings can be considered as problem settings for estimating parameters of the model proposed above from data. The first is setting 1 in which estimation is performed from input data including information regarding the number of steps. The second is setting 2 in which estimation is performed from input data including no information regarding the number of steps. Both will be described with reference to FIGS. 6 and 7. FIG. 6 is a diagram when data including information regarding the number of steps is taken as an input. FIG. 7 is a diagram when data including no information regarding the number of steps is taken as an input.

An example of the data represented in the format illustrated in FIG. 6 is medical examination history data illustrated in FIG. 4. In the example of this data, one month is set as one step. Indeed, the number of steps of each transition is not provided in FIG. 6, but from information that “User A was normal in April but became medium in June,” it can be seen that this is the result of a transition of two months, that is, two steps. Thus, each piece of data represents information that “state i has transitioned to state j in k steps,” such that data can be expressed in the format illustrated in FIG. 6 by counting and listing the number of times the transition has occurred.

An example of data expressed in the format illustrated in FIG. 7 is movement history data illustrated in FIG. 2. In this data, even if information that “a movement has been made from state 9 to state 12” is given as shown by a dotted arrow, what number of steps have been transitioned through to result in this movement is not known, unlike in the case of setting 1. For example, it is not known which number of steps of a transition results in the movement among a great number of transitions such as those of a two-step movement of 9 →10 →12, a three-step movement of 9 →10 →11 →12, and a five-step movement of 9 →10 →11 →6 →5 →12. Thus, each piece of data represents only information that “a transition has occurred from state i to state j” and includes no information regarding the number of steps, such that data can be expressed in the format illustrated in FIG. 7 by counting and listing the number of times the transition has occurred.

Parameter estimation of the proposed model can be performed in either setting 1 or setting 2. The setting 1 and the setting 2 differ in the availability of the number of steps in input data. The setting 1 is a setting for the case where the number of steps is available for a set of transitions between states of input data. Setting 2 is a setting for the case where the number of steps is not available for a set of transitions between states of input data. In order to consider such a difference in input data, it is necessary to perform estimation using different objective functions in the two cases. The estimation for each setting is described below.

Setting 1: Parameter estimation using data in which the number of steps is available Input data is expressed as follows.

[symbol]

₁={Nᵢⱼₖ}_{i,j,∈x,k∈{1, . . . ,Kmax}}

Nᵢⱼₖ represents the number of times a k-step transition has occurred from state i to state j. A subscript is expressed as “⋅” in the sense that summation is performed over the subscript. For example, N_{i⋅k}=ΣⱼNᵢⱼₖ.

The proposed model has been modeled assuming that the above input data is generated as follows. Because the probability that a k-step transition occurs from each state i is f(k|λᵢ), the probability of generating N_{i⋅k} representing the number of times a k-step transition has occurred from the state i is given by the following expression (8).

[Math. 7]

Pr(N_{i⋅k}|λᵢ)=f(k|λᵢ)^{N}^{_{i⋅k}} (8)

Further, because the transition probability of a k-step transition is given by the transition probability of one step to the kth power according to Theorem 1, the probability that a k-step transition occurs from the state i to j Nᵢⱼₖ times is given by the following expression (9).

[Math. 8]

Pr(Nᵢⱼₖ|η)={(P_{η}^{k})ᵢⱼ}^{N}^{ᵢⱼₖ} (9)

Summarizing this, the generation probability of data D1 of the model is given by the following expression (10).

[Math. 9]

Pr( [symbol] ₁|θ)=(λ,η)=Π_{i,k}{Pr(N_{i⋅k}|λᵢ)ΠⱼPr(Nᵢⱼₖ|η} (10)

Thus, the following expression (11) can be used as an objective function by taking the negative logarithm of the generation probability and adding a regularization term to prevent parameters from diverging. Expression (11) is an example of a first objective function.

[Math. 10]

[symbol]

₁(θ)=−log Pr( [symbol] ₁|θ)+αΩ(θ) (11)

where α is a hyperparameter and Ω(θ) is a regularization term, for which any regularization term such as the L2 norm can be used. As described above, the first objective function is an objective function including a term in which the generation probability of input data is given by the product of the model regarding the number of steps and the product of the probabilities of the number of times a transition of a predetermined number of k steps occurs between states, the probabilities thereof being given by the model regarding the transition probability, as shown in expression (10). Optimizing this objective function can obtain an estimated value ∧θ of the parameter of expression (12) below. The optimization method will be described later.

$$\begin{matrix}[ Math . 11 ] & \\ \hat{θ} = {arg min}_{θ} {ℒ}_{1} ( θ ) . & ( 12 )\end{matrix}$$

Setting 2: Parameter estimation using data in which the number of steps is not available Input data is expressed as follows.

[symbol]

₂={Nᵢⱼ}_{i,j∈}x

Nᵢⱼ represents the number of times a transition has occurred from state i to state j. Unlike in setting 1, there is no information regarding the number of steps.

The proposed model has been modeled assuming that the data is generated as follows. Because the probability that a k-step transition occurs from each state i is f(k|λᵢ) and the transition probability of a k-step transition is given by the transition probability of one step to the kth power according to Theorem 1, the following expression (13) is the generation probability of Nᵢⱼ.

[Math. 12]

Pr(Nᵢⱼ|θ)=(Σₖf(k|λᵢ)(P_{η}^{k})ᵢⱼ)^{N}^{ᵢⱼ} (13)

Therefore, the generation probability of data D2 of the model is given by the following expression (14).

[Math. 13]

Pr( [symbol] ₂|θ)=(λ,η))=Π_{i,j}{(Σₖf(k|λᵢ)(P_{η}^{k})ᵢⱼ)^{N}^{ᵢⱼ}} (14)

Thus, the following expression (15) can be used as an objective function by taking the negative logarithm of the generation probability and adding a regularization term to prevent parameters from diverging. Expression (15) is an example of a second objective function.

[Math. 14]

[symbol]

₂(θ)=−log Pr( [symbol] ₂|θ)+αΩ(θ) (15)

α and Ω(θ) are the same as in expression (11). As described above, the second objective function is an objective function including a term in which the generation probability of input data is given by the product of the model regarding the number of steps and the model regarding the transition probability as shown in expression (14). Optimizing this objective function can obtain an estimated value ∧θ of the parameter of expression (16) below.

$$\begin{matrix}[ Math . 15 ] & \\ \hat{θ} = {arg min}_{θ} {ℒ}_{2} ( θ ) . & ( 16 )\end{matrix}$$

Optimization Method

Next, the optimization method will be described. Here, an objective function L1 for the setting 1 and an objective function L2 for the setting 2 will be collectively denoted by L because the optimization method is common to both the setting 1 and the setting 2. Any optimization method such as a gradient method or Newton's method can be applied to the optimization of the objective function. When the gradient method is used, parameter update is repeated according to the following expression (8) in a qth optimization step.

[Math. 16]

θ_{q+1}←θ_{q}−γ_{q}∇_{θ} [symbol] (θ_{q}) (17)

where γ_{q} is a learning rate parameter. For the gradient ∇_{θ}L(θ) of the objective function, a function derived by computation may be used or a numerical computation method may be used.

An expectation-maximization (EM) algorithm can also be used as a mode limited to the setting 2. This is because expression (14) can be regarded as a mixed distribution of transition probabilities P_{η}^{k} having f(k|λᵢ) as a mixture ratio. An algorithm that introduces the following latent variable that is not actually observed can be created.

{Mᵢⱼₖ}_{i,j∈x,k∈{1, . . . ,Kmax}}

where it is assumed that Mᵢⱼₖ represents the number of times a k-step transition has occurred from state i to state j and that Mᵢⱼ =Nᵢⱼ is satisfied. An algorithm that repeatedly updates the latent variable {Mᵢⱼₖ} and the parameter θ can be created in this manner.

The parameter estimation apparatus of the present disclosure optimizes parameters using the above objective function and optimization method.

Hereinafter, a configuration of the present embodiment will be described.

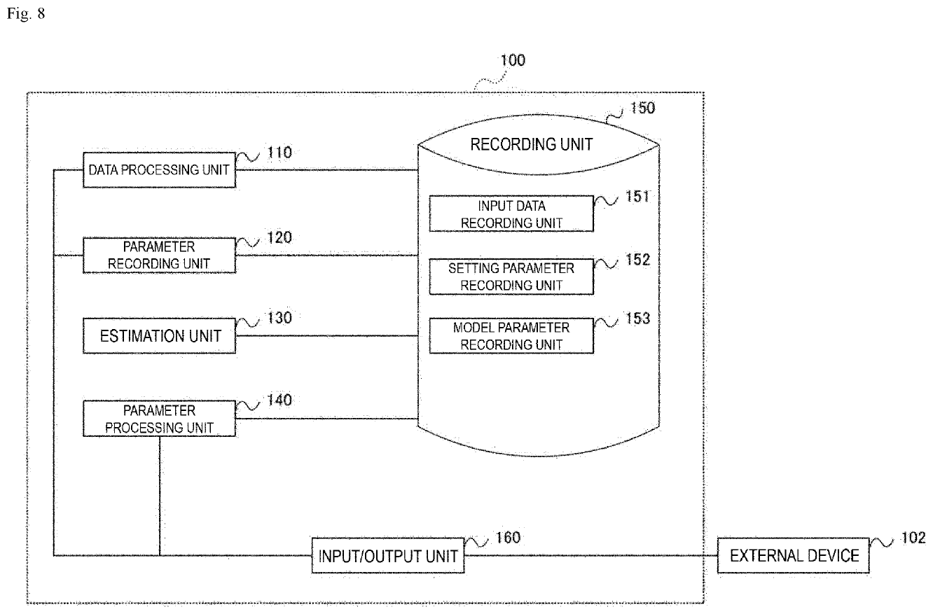

FIG. 8 is a block diagram illustrating a configuration of the parameter estimation apparatus of the present embodiment.

As illustrated in FIG. 8, the parameter estimation apparatus 100 includes a data processing unit 110, a parameter recording unit 120, an estimation unit 130, a parameter processing unit 140, a recording unit 150, and an input/output unit 160. The parameter estimation apparatus 100 is connected to an external device 102 via a network (not illustrated) and various data is transmitted and received through the input/output unit 160.

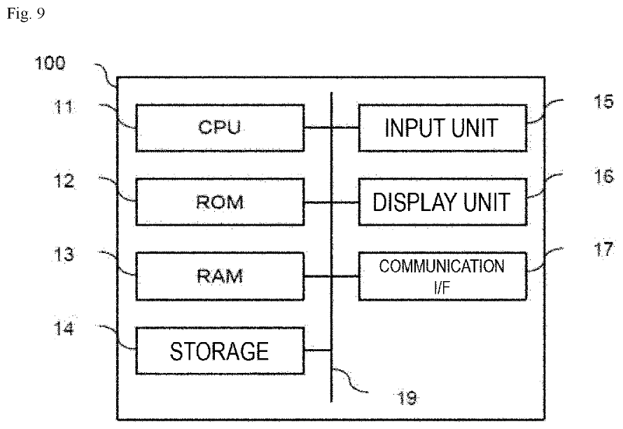

FIG. 9 is a block diagram illustrating a hardware configuration of the parameter estimation apparatus 100.

As illustrated in FIG. 9, the parameter estimation apparatus 100 includes a central processing unit (CPU) 11, a read only memory (ROM) 12, a random access memory (RAM) 13, a storage 14, an input unit 15, a display unit 16, and a communication interface (I/F) 17. These components are communicatively connected to each other via a bus 19.

The CPU 11 is a central arithmetic processing unit and executes various programs and controls each part. That is, the CPU 11 reads a program from the ROM 12 or the storage 14 and executes the program using the RAM 13 as a work area. The CPU 11 controls each of the components described above and performs various arithmetic processing according to programs stored in the ROM 12 or the storage 14. In the present embodiment, the ROM 12 or the storage 14 stores a parameter estimation program.

The ROM 12 stores various programs and various data. The RAM 13 is a work area that temporarily stores a program or data. The storage 14 includes a storage device such as a hard disk drive (HDD) or a solid state drive (SSD) and stores various programs including an operating system and various data.

The input unit 15 includes a pointing device such as a mouse and a keyboard and is used to perform various inputs.

The display unit 16 is, for example, a liquid crystal display and displays various types of information. The display unit 16 may adopt a touch panel method and function as an input unit 15.

The communication interface 17 is an interface for communicating with other devices such as a terminal and uses standards such as, for example, Ethernet (registered trademark), FDDI, and Wi-Fi (registered trademark).

Next, each functional component of the parameter estimation apparatus 100 will be described. Each functional component is realized by the CPU 11 reading the parameter estimation program stored in the ROM 12 or the storage 14 and loading and executing the parameter estimation program into and from the RAM 13.

The input/output unit 160 receives input data and setting parameters of the objective function from the external device 102.

The data processing unit 110 records the input data received by the input/output unit 160 in an input data recording unit 151 in the recording unit 150. The input data is the input data D₁ or input data D₂ described above.

The parameter recording unit 120 records the setting parameters received by the input/output unit 160 in a setting parameter recording unit 152 in the recording unit 150. The setting parameters are a hyperparameter α of the objective function, information [Ωᵢ]_{i∈X} regarding a set of states reachable from each state, a learning rate parameter γ_{q}, and the like.

The estimation unit 130 reads the input data recorded in the input data recording unit 151 and the setting parameters recorded in the setting parameter recording unit 152, executes a parameter estimation process, and records estimated parameters θ=(η,λ) in a model parameter recording unit 153.

As a process, the estimation unit 130 estimates the parameters θ=(η,λ) such that the objective function represented by the above expression (11) or (15) is optimized. η is a parameter relating to the model regarding the transition probability representing the probability that a one-step transition occurs from each state. λ is a parameter relating to the model regarding the number of steps representing the probability that a transition of a predetermined number of steps occurs from each state. In the optimization method for estimation, a process of estimating the parameters θ according to the above expression (17) is repeated until a predetermined condition is satisfied. For example, the maximum number of repetitions is set as a predetermined condition.

The parameter processing unit 140 transmits the parameters θ recorded in the model parameter recording unit 153 to the external device 102 through the input/output unit 160.

Next, an operation of the parameter estimation apparatus 100 will be described.

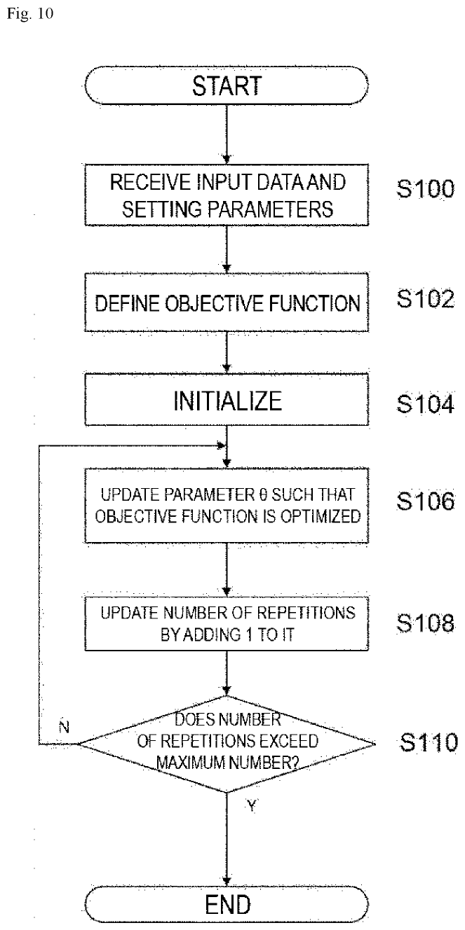

FIG. 10 is a flowchart showing the flow of a parameter estimation process performed by the parameter estimation apparatus 100. The parameter estimation process is performed by the CPU 11 reading the parameter estimation program from the ROM 12 or the storage 14 and loading and executing the parameter estimation program into and from the RAM 13.

In step S100, the CPU 11 receives the input data and the setting parameters as inputs and records them in the respective recording units of the recording unit 150 as described above. The CPU 11 receives D₁ or D₂ as input data and records it in the input data recording unit 151. The CPU 11 receives a hyperparameter α of the objective function, information [Ωᵢ]_{i∈X} regarding a set of states reachable from each state, a learning rate parameter γ_{q}, and the like as setting data and records them in the setting parameter recording unit 152.

In step S102, the CPU 11 reads the input data from the input data recording unit 151, reads the setting parameters from the setting parameter recording unit 152, and defines an objective function, for example, as shown in expression (11) or (15). If the input data is input data of the setting 1, the CPU 11 defines an objective function as shown in expression (11). If the input data is input data of the setting 2, the CPU 11 defines an objective function as shown in expression (15).

In step S104, the CPU 11 initializes the parameters θ, sets the number of repetitions q such that q=0, and sets the maximum number of repetitions Q.

In step S106, the CPU 11 updates and estimates the parameters θ according to the above expression (17) such that the objective function defined in step S102 is optimized.

In step S108, the CPU 11 updates the number of repetitions q by adding 1 to the number of repetitions q.

In step S110, the CPU 11 determines whether or not the number of repetitions q exceeds the maximum number Q. If the number of repetitions q exceeds the maximum number Q, the CPU 11 records the estimation result of the parameters θ in the model parameter recording unit 153 and ends the process. If the number of repetitions q does not exceed the maximum number Q, the CPU 11 returns to step S106 and repeats the process.

The parameter estimation apparatus 100 of the present embodiment can accurately estimate parameters of a Markov chain using transition data whose observation interval is not constant as described above.

Although the above embodiment has been described with respect to the case where the objective function of expression (11) or expression (15) is used, the present disclosure is not limited thereto. For example, there are cases where input data is in D₁ and D₂ formats and data in the two formats can be obtained. That is, there may be a case where a set of transitions between states of input data includes transitions where the number of steps is available and transitions where the number of steps is not available. In this case, estimation is performed using a third objective function including a term that sums the first objective function of expression (11) and the second objective function of expression (15) for the input data D₁ and D₂.

Although the above embodiment shows an example in which the gradient method is used for optimization, any method such as Newton's method can be used. Similarly, any models can be used as those for the state transition probability and the initial state probability. Similarly, any regularization term can be used as that of the objective function. Further, the parameter estimation apparatus illustrated in FIG. 8 of the above embodiment may be implemented such that the operation of each component is constructed as a program and then installed on and executed by a computer used as the parameter estimation apparatus or distributed via a network. The present disclosure is not limited to the above embodiments and various modifications and applications are possible.

The parameter estimation process executed by the CPU reading software (program) in the above embodiment may also be executed by various processors other than the CPU. Examples of such processors include a programmable logic device (PLD) whose circuit configuration can be changed after manufacturing such as a field-programmable gate array (FPGA) and a dedicated electric circuit which is a processor having a circuit configuration specially designed to execute specific processing such as an application specific integrated circuit (ASIC). The parameter estimation process may be executed by one of these various processors or may be executed by a combination of two or more processors of the same type or different types (such as, for example, a plurality of FPGAs or a combination of a CPU and an FPGA). A hardware structure of these various processors is, more specifically, an electric circuit that combines circuit elements such as semiconductor elements.

The above embodiments have been described with reference to a mode in which the parameter estimation program is stored (installed) in the storage 14 in advance. However, the present disclosure is not limited to this. Programs may be provided in a form stored in a non-transitory storage medium such as a compact disc read only memory (CD-ROM), a digital versatile disc ROM (DVD-ROM), or a universal serial bus (USB) memory. Programs may also be in a form downloaded from an external device via a network.

Regarding the above embodiments, the following supplements are further disclosed.

Supplement 1

A parameter estimation apparatus including:
a memory; and
at least one processor connected to the memory,
wherein the processor is configured to, assuming that transition intervals of a Markov chain defined from a set of states are steps, receive input data that is transition data including the number of transitions between states in a set of transitions between states and estimate a parameter relating to a model regarding the number of steps representing a probability that a transition of a predetermined number of steps occurs from each state and a parameter relating to a model regarding a transition probability representing a probability that a one-step transition occurs from each state, the models being models for the number of transitions between the states of the input data, such that an objective function including a term for a generation probability of the input data given by the model regarding the number of steps and the model regarding the transition probability is optimized.

Supplement 2

A non-transitory storage medium storing a parameter estimation program causing a computer to, assuming that transition intervals of a Markov chain defined from a set of states are steps, receive input data that is transition data including the number of transitions between states in a set of transitions between states and estimate a parameter relating to a model regarding the number of steps representing a probability that a transition of a predetermined number of steps occurs from each state and a parameter relating to a model regarding a transition probability representing a probability that a one-step transition occurs from each state, the models being models for the number of transitions between the states of the input data, such that an objective function including a term for a generation probability of the input data given by the model regarding the number of steps and the model regarding the transition probability is optimized.

### REFERENCE SIGNS LIST

100 Parameter estimation apparatus
102 External device
110 Data processing unit
120 Parameter recording unit
130 Estimation unit
140 Parameter processing unit
150 Recording unit
151 Input data recording unit
152 Setting parameter recording unit
153 Model parameter recording unit
160 Input/output unit

1. A parameter estimation apparatus comprising a circuit configured to execute a method comprising:
receiving, based on transition intervals of a Markov chain being defined from a set of states are steps, input data, wherein the input data include transition data representing the number of transitions between states in a set of transitions between states; and
estimating a parameter relating to a model regarding a number of steps representing a probability that a transition of a predetermined number of steps occurs from each state and another parameter relating to a model regarding a transition probability representing a probability that a one-step transition occurs from each state, such that an objective function including a term for a generation probability of the input data given by the model regarding the number of steps and the other model regarding the transition probability is optimized.

2. The parameter estimation apparatus according to claim 1,
wherein the objective function includes either:
a first objective function including a term in which the generation probability of the input data is given by a product of the model regarding the number of steps and a product of probabilities of a number of times a transition of a predetermined number of steps occurs between states, the probabilities thereof being given by the model regarding the transition probability, when the set of the transitions between the states of the input data includes transitions in which the number of steps is available, or
a second objective function including a term in which the generation probability of the input data is given by a product of the model regarding the transition probability and the model regarding the number of steps when the set of the transitions between the states of the input data includes transitions in which the number of steps is not available.

3. The parameter estimation apparatus according to claim 2, wherein the objective function includes a third objective function including a term that sums the first objective function and the second objective function when the set of the transitions between the states of the input data includes transitions in which the number of steps is available and transitions in which the number of steps is not available.

4. A computer-implemented method for estimating parameters, the method comprising:
receiving, based on transition intervals of a Markov chain being defined from a set of states are steps, input data, wherein the input data represent transition data including the number of transitions between states in a set of transitions between states; and
estimating a parameter relating to a model regarding number of steps representing a probability that a transition of a predetermined number of steps occurs from each state and a parameter relating to another model regarding a transition probability representing a probability that a one-step transition occurs from each state, such that an objective function including a term for a generation probability of the input data given by the model regarding the number of steps and the other model regarding the transition probability is optimized.

5. The computer-implemented method according to claim 4, wherein the objective function includes either:
a first objective function including a term in which the generation probability of the input data is given by a product of the model regarding the number of steps and a product of probabilities of a number of times a transition of a predetermined number of steps occurs between states, the probabilities thereof being given by the model regarding the transition probability, when the set of the transitions between the states of the input data includes transitions in which the number of steps is available, or
a second objective function including a term in which the generation probability of the input data is given by a product of the model regarding the transition probability and the model regarding the number of steps when the set of the transitions between the states of the input data includes transitions in which the number of steps is not available.

6. The computer-implemented method according to claim 5, wherein the objective function includes a third objective function including a term that sums the first objective function and the second objective function when the set of the transitions between the states of the input data includes transitions in which the number of steps is available and transitions in which the number of steps is not available.

7. A computer-readable non-transitory recording medium storing computer-executable program instructions that when executed by a processor cause a computer system to execute a method comprising:
receiving, based on transition intervals of a Markov chain being defined from a set of states are steps, input data, wherein the input data represent transition data including the number of transitions between states in a set of transitions between states; and
estimating a parameter relating to a model regarding a number of steps representing a probability that a transition of a predetermined number of steps occurs from each state and a parameter relating to another model regarding a transition probability representing a probability that a one-step transition occurs from each state, such that an objective function including a term for a generation probability of the input data given by the model regarding the number of steps and the other model regarding the transition probability is optimized.

8. The parameter estimation apparatus according to claim 1, wherein the transition data represent movement histories of people in areas, including data associated with the areas and a period of staying in an area greater than a predetermined time period.

9. The parameter estimation apparatus according to claim 1, wherein the transition data represent medical treatment histories of patients, including probability of symptoms of a disease and a frequency of observation.

10. The computer-implemented method according to claim 4, wherein the transition data represent movement histories of people in areas, including data associated with the areas and a period of staying in an area greater than a predetermined time period.

11. The computer-implemented method according to claim 4, wherein the transition data represent medical treatment histories of patients, including probability of symptoms of a disease and a frequency of observation.

12. The computer-readable non-transitory recording medium according to claim 7, wherein the objective function includes either:
a first objective function including a term in which the generation probability of the input data is given by a product of the model regarding the number of steps and a product of probabilities of a number of times a transition of a predetermined number of steps occurs between states, the probabilities thereof being given by the model regarding the transition probability, when the set of the transitions between the states of the input data includes transitions in which the number of steps is available, or
a second objective function including a term in which the generation probability of the input data is given by a product of the model regarding the transition probability and the model regarding the number of steps when the set of the transitions between the states of the input data includes transitions in which the number of steps is not available.

13. The computer-readable non-transitory recording medium according to claim 12, wherein the objective function includes third objective function including a term that sums the first objective function and the second objective function when the set of the transitions between the states of the input data includes transitions in which the number of steps is available and transitions in which the number of steps is not available.

14. The computer-readable non-transitory recording medium according to claim 7, wherein the transition data represent movement histories of people in areas, including data associated with the areas and a period of staying in an area greater than a predetermined time period.

15. he computer-readable non-transitory recording medium according to claim 7, wherein the transition data represent medical treatment histories of patients, including probability of symptoms of a disease and a frequency of observation.
